(19) United States
(12) Patent Application Publication
(10) Pub. No.: US 2004/0005357 A1
(43) Pub. Date: Jan. 8, 2004

(54) Extended-release tablets comprising divalproex sodium

(76) Inventor: Bernard Charles Sherman, Toronto (CA)

Correspondence Address: NIXON & VANDERHYE, PC, 1100 N GLEBE ROAD, 8TH FLOOR, ARLINGTON, VA, 22201-4714, US

(21) Appl. No.: 10/187,908

(22) Filed: Jul. 3, 2002

(51) Int. Cl. A61K 9/20; A61K 9/22; A61K 31/19

(52) U.S. Cl. 424/468; 514/557

(57) ABSTRACT

Extended-release tablets comprising divalproex sodium and a water-insoluble polymer wherein the amount of divalproex sodium exceeds 80% of the tablet by weight.

### BACKGROUND OF THE INVENTION

Divalproex sodium is a valproic acid derivative useful as an antiepileptic agent.

Delayed-release tablets comprising divalproex sodium in strengths of 125 mg, 250 mg and 500 mg are sold in the United States and elsewhere under the tradename Depakote™. The delay in release is achieved by coating the tablets with an enteric coating, which is a coating comprising a polymer that is insoluble in aqueous media at gastric (i.e. acidic) pH, but dissolves in the more basic intestinal fluids. The tablets thus are protected by the enteric coating until they reach the small intestine, at which point the coating dissolves and the tablet cores disintegrate to release the divalproex sodium. The coating thus prevents the divalproex sodium from causing gastric irritation. ™—Trademark

After absorption through the intestinal mucosa into systemic circulation, the elimination half life of divalproex sodium is relatively short, so that blood levels fluctuate substantially between doses.

To overcome this problem, extended-release divalproex sodium tablets are now sold in the United States and elsewhere under the tradename Depakote ER™.

Each tablet contains 540 mg of divalproex sodium, which is equivalent to 500 mg of valproic acid. As with Depakote™ tablets, these tablets consist of core tablets, which are coated with a film coating. However, in the case of Depakote ER™ tablets, the core tablets do not disintegrate promptly in intestinal fluid, but erode away only very slowly, so that the divalproex sodium is released very gradually over a period of many hours.

According to the labelling, the inactive ingredients in the core tablets include hydroxypropyl methylcellulose and microcrystalline cellulose.

Hydroxypropyl methylcellulose is known to be gel-forming polymer; that is to say, in aqueous media it absorbs water to form a viscous gel which dissolves slowly. It thus appears that hydroxypropyl methylcellulose is the principle ingredient used in Depakote ER™ tablets to achieve extended release. Core tablets comprising only divalproex sodium and hydroxypropyl methylcellulose would be too soft to withstand the film coating process. Microcrystalline cellulose is thus included in Depakote ER™ tablets for the purpose of increasing tablet hardness.

Depakote ER™ tablets are made in accordance with the teachings of U.S. Pat. No. 4,913,906. This patent teaches a controlled (i.e. extended) release tablet comprising valproic acid or a derivative thereof as active ingredient and a polymer for achieving extended release, wherein the active ingredient comprises from 10 to 80 percent of the tablet by weight.

Each Depakote ER™ tablet contains 540 mg of divalproex sodium. Each tablet weighs about 1050 mg. The weight of the film coating is about 30 mg, so that the weight of the core tablet is about 1020 mg. Divalproex sodium thus comprises about 540/1020 or 52.9% of the core tablet by weight.

Because 1050 mg is a relatively high weight for a pharmaceutical tablet, Depakote ER™ tablets are relatively large in size, and thus relatively difficult to swallow. The relatively large size also causes the cost of manufacture to be higher than would be the case for a smaller tablet.

In light of this prior art, an objective of the present invention is to enable extended-release tablets comprising divalproex sodium which are substantially smaller than Depakote ER™ tablets having the same content of divalproex sodium.

### DESCRIPTION OF THE INVENTION

It has been found that it is possible to make extended-release tablets which comprise divalproex sodium and a water-insoluble polymer, which have a dissolution rate comparable to that of Depakote ER™, and which also have sufficient hardness to withstand a film-coating process, without the need to include microcrystalline cellulose to increase tablet hardness. A divalproex sodium content above 80 percent by weight can thus be achieved.

Tablets of the present invention are thus extended-release tablets, which comprise divalproex sodium, and a water-insoluble polymer, wherein the divalproex sodium comprises more than 80 percent of the tablet by weight. The tablets will preferably be made by a process in which the divalproex sodium and polymer are wetted by a solvent, which may be water or a volatile organic solvent, and the solvent is thereafter evaporated.

As aforesaid, the tablets will comprise divalproex sodium and a water-insoluble polymer. The amount of divalproex sodium will preferably be from 85% to 98% of the tablet by weight, more preferably from 85% to 95% and most preferably about 90%.

Suitable water-insoluble polymers will include, for example, ethylcellulose, cellulose acetate, polyvinyl acetate, and methacrylic ester copolymers. Most preferred is ethylcellulose.

The amount of the water-insoluble polymer will preferably be from 2% to 18% of the tablet by weight, more preferably from 5% to 15%, and most preferably about 10%.

As aforesaid, the process of manufacture will preferably be one in which the divalproex sodium and polymer are wetted by water or an organic solvent, and the solvent is thereafter evaporated.

If water is used, the polymer will preferably be used in the form of a latex dispersion of the polymer, and optionally a plasticizer, in the water. The latex dispersion will be mixed into the divalproex sodium, and the mixture then dried to evaporate the water.

Preferably, the process will not use water, but will use a volatile organic solvent in which the polymer is soluble. The process may be carried out either by first mixing the divalproex sodium and polymer in dry form and then mixing the solvent into the powder mixture, or by first dissolving the polymer in the solvent and then mixing the solution into the divalproex sodium. Preferred solvents are lower alcohols such as methanol, and chlorinated hydrocarbons such as methylene chloride. Again, optionally a plasticizer may also be included along with the polymer. Again, the wet mass is then dried to evaporate the solvent.

The dried mass then milled into free-flowing granules, which are then compressed into tablets on a tablet press.

Optionally, other inactive ingredients, such as a lubricant or glidant will be mixed with the granules before compression into tablets. However, it has been found that no such other ingredients are required, and, more particularly, that tablets can be made that have adequate hardness for film coating without addition of microcrystalline cellulose. Hence, it is preferred to directly compress the granules into tablets, without addition of other inactive ingredients.

Because divalproex sodium has a foul taste, the tablets will then preferably have a film coating applied to cover the taste. Polymer systems and processes for film-coating of tablets are well known in the art. In the case of film-coated tablets, the tablet weight used for calculation of the percentage content of divalproex sodium and the water-insoluble polymer will be understood to refer to the weight of the core tablet only, exclusive of the weight of the film coating.

The film coating may comprise a water-soluble polymer. However, the film coating will preferably be an enteric coating which will be understood to mean a coating which is insoluble at gastric (acidic) pH but which dissolves at the more basic intestinal pH. An enteric-coating is preferable both because it is more effective in covering the taste of the core and it prevents release of the divalproex sodium in the stomach and thus prevents both gastric irritation and food effect. Food effect will be understood to mean an increased rate of absorption upon emptying of the stomach into the small intestine, as a result of dissolution in the stomach during the extended period of time that the tablet will reside in the stomach in the fed state.

The invention will be better understood from the following example, which is intended to be illustrative and not limiting of the scope of the invention.

### EXAMPLE 1

Ingredients were used in the following proportions:

| | |
|---|---|
| Divalproex Sodium | 90. |
| Ethylcellulose 10CPS | 10. |
| Methylene Chloride | 50. |

The ethylcellulose was dissolved in the methylene chloride. The solution was then added to and mixed into the divalproex sodium. The wet mass was then dried and milled into granules.

The granules were then compressed into tablets of weight 600 mg. Each tablet thus contained 540 mg of divalproex sodium. The tablets have hardness more than adequate to enable film-coating.

The dissolution rate of the resultant tablets was then compared to that of Depakote ER™ tablets.

The apparatus used was United States Pharmacopoeia apparatus no. 2, at 100 rpm. The dissolution medium was 900 mL of phosphate buffer pH 6.8. For both the tablets of example 1 and Depakote ERTM tablets, the extent of dissolution was about 50% at 12 hours and about 70% at 24 hours.

1. An extended release tablet comprising divalproex sodium and a water-insoluble polymer, wherein the divalproex sodium comprises more than 80 percent of the tablet by weight.

2. A tablet of claim 1, wherein the amount of divalproex sodium is from 82 percent to 98 percent of the tablet by weight.

3. A tablet of claim 2, wherein the amount of divalproex sodium is from 85 percent to 95 percent of the tablet by weight.

4. A tablet of claim 3, wherein the amount of divalproex sodium is about 90 percent of the tablet by weight.

5. A tablet of any of claims 1 to 4, wherein the polymer is selected from ethylcellulose, cellulose acetate, and polyoxyl acetate and methacrylic ester copolymer.

6. A tablet of claim 5, wherein the polymer is ethylcellulose.

7. A tablet of any of claims 1 to 6, wherein the amount of the polymer is from 2 percent to 18 percent of the tablet by weight.

8. A tablet of claim 7, wherein the amount of the polymer is from 5 percent to 15 percent of the tablet by weight.

9. A tablet of claim 8, wherein the amount of the polymer is about 10% of the tablet by weight.

10. A tablet of any of claims 1 to 9, when made by a process in which the divalproex sodium and polymer are wetted by water or a volatile organic solvent and the water or organic solvent is thereafter evaporated.

11. A tablet of claim 10, wherein the polymer is used in the form of a latex dispersion in water.

12. A tablet of claim 10, when made by a process in which the divalproex sodium and polymer are mixed together in dry form, the mixture is wetted with a volatile organic solvent in which the polymer is soluble, and the solvent is then evaporated.

13. A tablet of claim 10, when made by a process in which the polymer is dissolved in a volatile organic solvent, the solution is added to and mixed into the divalproex sodium, and the solvent is then evaporated.

14. A tablet of claim 12 or 13 when the solvent is a lower alcohol.

15. A tablet of claim 14 wherein the solvent is methanol.

16. A tablet of claim 12 or 13 wherein the solvent is a chlorinated hydrochloride.

17. A tablet of claim 16 wherein the solvent is methylene chloride.

18. A tablet of any of claims 1 to 17, which consists of only divalproex sodium, a water-insoluble polymer, and optionally a plasticizer, and no other ingredients.

19. A tablet of any of claims 1 to 17, which is free of microcrystalline cellulose.

20. A tablet of any of claims 1 to 19, which is enteric coated.
